(19) United States
(12) Patent Application Publication
(10) Pub. No.: US 2024/0410723 A1
(43) Pub. Date: Dec. 12, 2024

(54) PULSER WITH DOUBLE-BEARING POSITION ENCODER FOR NON-INVASIVE PHYSIOLOGICAL MONITORING

(71) Applicant: Masimo Corporation, Irvine, CA (US)

(72) Inventor: Cristiano Dalvi, Lake Forest, CA (US)

(21) Appl. No.: 18/638,084

(22) Filed: Apr. 17, 2024

Related U.S. Application Data
(63) Continuation of application No. 17/324,474, filed on May 19, 2021, now Pat. No. 11,988,532.
Application No. 17/324,474 is a continuation of application No. 15/847,579, filed on Dec. 19, 2017, now Pat. No. 11,022,466.
Application No. 15/847,579 is a continuation of application No. 14/334,662, filed on Jul. 17, 2014, now Pat. No. 9,891,079.
(60) Provisional application No. 61/847,307, filed on Jul. 17, 2013.

(51) Int. Cl. G01D 5/347; A61B 5/1455; G01D 11/02

(52) U.S. Cl. CPC G01D 5/34738; A61B 5/1455; G01D 11/02

(57) ABSTRACT

A double-bearing position encoder has an axle stabilized within a housing via two bearings disposed on opposite walls of the housing. The axle is in communications with a rotating cam. The cam actuates a pulser so as to generate an active pulse at a tissue site for analysis by an optical sensor. The axle rotates a slotted encoder wheel or a reflective encoder cylinder disposed within the housing so as to accurately determine the axle position and, hence, the active pulse frequency and phase.

### CROSS-REFERENCE TO RELATED APPLICATIONS

Any and all applications for which a foreign or domestic priority claim is identified above or in the Application Data Sheet as filed with the present application are incorporated by reference under 37 CFR 1.57 and made a part of this specification.

### BACKGROUND

Noninvasive physiological monitoring systems for measuring constituents of circulating blood have advanced from basic pulse oximeters to monitors capable of measuring abnormal and total hemoglobin among other parameters. A basic pulse oximeter capable of measuring blood oxygen saturation typically includes an optical sensor, a monitor for processing sensor signals and displaying results and a cable electrically interconnecting the sensor and the monitor. A pulse oximetry sensor typically has a red wavelength light emitting diode (LED), an infrared (IR) wavelength LED and a photodiode detector. The LEDs and detector are attached to a patient tissue site, such as a finger. The cable transmits drive signals from the monitor to the LEDs, and the LEDs respond to the drive signals to transmit light into the tissue site. The detector generates a photoplethysmograph signal responsive to the emitted light after attenuation by pulsatile blood flow within the tissue site. The cable transmits the detector signal to the monitor, which processes the signal to provide a numerical readout of oxygen saturation (SpO.sub.2) and pulse rate, along with an audible pulse indication of the person's pulse. The photoplethysmograph waveform may also be displayed.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, Calif. and are incorporated in their entireties by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entireties by reference herein. Advanced pulse oximetry systems including Masimo SET® low noise optical sensors and read through motion pulse oximetry monitors for measuring SpO.sub.2, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are described in at least U.S. Pat. No. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, Calif. (“Cercacor”) and all incorporated in their entireties by reference herein. An advanced parameter measurement system that includes acoustic monitoring is described in U.S. Pat. Pub. No. 2010/0274099, filed Dec. 21, 2009, titled Acoustic Sensor Assembly, assigned to Masimo and incorporated in its entirety by reference herein.

Advanced blood parameter measurement systems include Masimo Rainbow® SET, which provides measurements in addition to SpO.sub.2, such as total hemoglobin (SpHb™), oxygen content (SpOC™), methemoglobin (SpMet®), carboxyhemoglobin (SpCO®) and PVI® Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Advanced parameter measurement systems may also include acoustic monitoring such as acoustic respiration rate (RRa™) using a Rainbow Acoustic Sensor™ and Rad-87™ monitor, available from Masimo. Such advanced pulse oximeters, low noise sensors and advanced parameter systems have gained rapid acceptance in a wide variety of medical applications, including surgical wards, intensive care and neonatal units, general wards, home care, physical training, and virtually all types of monitoring scenarios.

FIG. 1 illustrates an active pulse generator 100 that installs within a reusable optical sensor for precisely pulsing a tissue site, such a fingertip. The active pulse generator 100 has a motor 110, a cam 120, a housing 130, a pulser 140 and an optical encoder 200. The cam 120 and pulser 140 are located within the housing 130. A shaft 160 couples the motor 110 to the cam 120 so as to linearly-actuate the pulser 140 upon application of electric current to the motor 110. The encoder 200 extends into the housing 130 so as to mechanically couple to the cam 120. The encoder 200 measures the rotation of the cam 120 and hence the position of the pulser 140. Based upon encoder feedback, the pulser 140 frequency and phase, and hence that of an active pulse, can be accurately measured and controlled. An active pulse reusable optical sensor is described in U.S. patent application Ser. No. 13/473,477, titled Personal Health Device, filed May 16, 2012 and assigned to Cercacor is hereby incorporated in its entirety by reference herein.

FIG. 2 further illustrates the encoder 200, which has a housing 210, a single-bearing 220 that mounts an encoder axle 230 to an encoder wheel 240 and an optics assembly that senses reflective position tracks and an index track on the encoder wheel 240 so as to generate a two-channel quadrature square wave output indicative of the axle 230 position.

### SUMMARY

A single-bearing encoder wheel mount, as described with respect to FIG. 2, above, has insufficient mechanical stability to provide optimum accuracy in measuring and controlling the phase and frequency of an optical sensor active pulse. Double-bearing position encoder embodiments advantageously improve encoder wheel stability so as to improve active pulse accuracy and also solve encoder wheel/optical reader configuration issues created by the necessary location of the stabilizing second bearing.

One aspect of a double-bearing position encoder is a housing, a pair of bearings disposed within opposite facing walls of the housing and an axle disposed within the housing and supported by the bearings. The axle is in mechanical communications with a pulser. An encoder wheel having wheel slots is fixedly attached to the axle. An LED is disposed within the housing so as to illuminate the encoder wheel. A detector is responsive to the LED illumination after optical interaction with the wheel slots as the axle rotates the wheel so as to indicate the wheel position.

In an embodiment, the axle is stabilized within a housing via bearings disposed on opposite walls of the housing. The axle is in communications with a rotating cam that actuates a pulser so as to generate an active pulse at a tissue site for analysis by an optical sensor. The axle rotates a slotted encoder wheel or a reflective encoder cylinder so as to accurately determine the axle position and, hence, the active pulse frequency and phase.

In various embodiment, the encoder comprises an encoder mask having mask slots disposed over an edge and along both sides of the encoder wheel so that the LED illumination passes through the mask slots and the wheel slots before reaching the detector. The encoder mask is folded so that LED light is reflected off of the mask a first time before illuminating the encoder wheel and second time before reaching the detector. Alternatively, the encoder mask is folded so that LED light is not reflected off of the mask before illuminating the encoder wheel and before reaching the detector.

Another aspect of a double-bearing position encoder is a rotatable axle. An encoder wheel is rotatably mounted on the double-bearing-mounted axle. An encoder mask is folded proximate an outer edge of the encoder wheel. Wheel slots are disposed around the encoder wheel proximate the outer edge. Mask slots are disposed through the encoder mask, and an emitter and a detector are disposed proximate to and on either side of the encoder wheel so that light intermittently passes through the encoder wheel via the wheel slots and the mask slots.

In various embodiments, light is reflected from the emitter off of the mask at least once before it reaches the detector. Light is reflected from the emitter off of the mask twice before it reaches the detector. The emitter directly illuminates the detector without reflection off the mask.

A further aspect of a double-bearing position encoder is a double bearing means of stabilizing a rotatable axle within an encoder housing. An encoder wheel means fixedly mounted to the axle so as to rotate as the axle rotates. An illumination and detection means of intermittently passing light through the encoder wheel means as it rotates, and a folded and slotted mask means of precisely passing light through encoder wheel.

### BRIEF DESCRIPTION OF THE DRAWINGS

FIG. 1 is a perspective illustration of an optical sensor active pulse generator including a single-bearing position encoder;

FIG. 2 is a cutaway side view of a single-bearing position encoder;

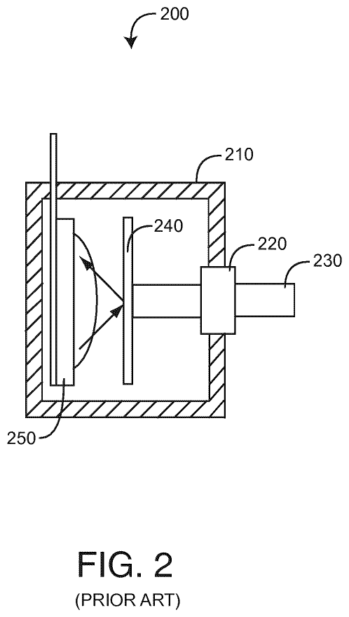

FIGS. 3A-B are cutaway side views of double-bearing position encoder embodiments incorporating a slotted wheel encoder;

FIG. 4 is a cutaway side view of a double-bearing position encoder embodiment incorporating a reflective cylinder encoder;

FIGS. 5A-B are front and back perspective views of a double-bearing position encoder assembly;

FIGS. 6A-B are partially exploded and exploded perspective views, respectively, of a double-bearing position encoder assembly;

FIGS. 7A-E are top, front, bottom, side and perspective views, respectively, of an encoder mask block;

FIGS. 8A-D are top, perspective, front and side views, respectively, of an encoder mask;

FIGS. 9A-D are top, perspective, front and side views, respectively, of a slotted encoder wheel;

FIGS. 10A-E are top, perspective, front, back and side views, respectively, of an encoder front housing;

FIGS. 11A-E are top, perspective, front, back and side views, respectively, of an encoder back housing;

FIGS. 12A-E are top, bottom, perspective, front and side views, respectively, of an encoder flex circuit;

FIGS. 13A-B are top and bottom exploded views, respectively, of flex circuit optics and a corresponding encoder mask block;

FIGS. 14A-B are assembled and partially exploded perspective views, respectively, of another double-bearing position encoder assembly;

FIGS. 15A-D are front, perspective, top and side views, respectively, of an encoder mask block;

FIGS. 16A-D are front, perspective, top and side views, respectively, of an encoder mask;

FIGS. 17A-B are top and bottom exploded views, respectively, of flex circuit optics and a corresponding encoder mask block;

FIGS. 18A-B are front and back perspective views of a further double-bearing position encoder assembly;

FIGS. 19A-B are top and bottom partially exploded perspective views, respectively, of a further double-bearing position encoder assembly;

FIGS. 20A-B are top mostly exploded and exploded perspective views, respectively, of a further double-bearing position encoder assembly;

FIGS. 21A-B are front and perspective views, respectively, of a first encoder cylinder embodiment;

FIGS. 22A-B are front and perspective views, respectively, of a second encoder cylinder embodiment; and

FIGS. 23A-B are front and perspective views, respectively, of a third encoder cylinder embodiment.

### DETAILED DESCRIPTION

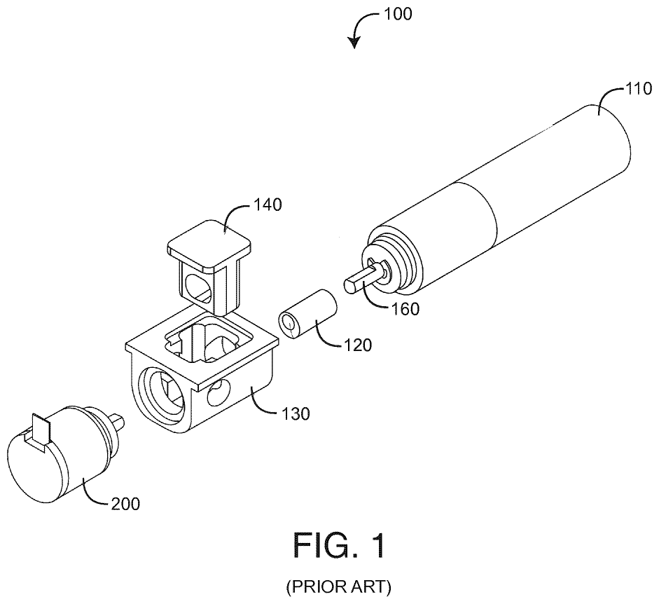

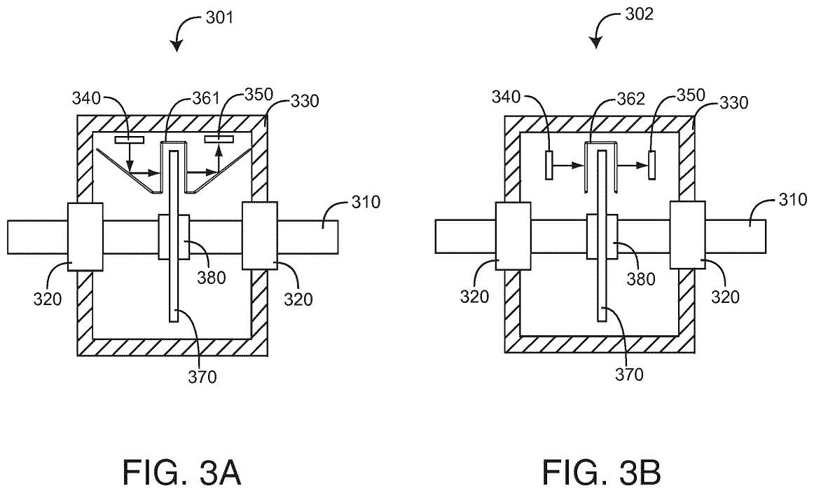

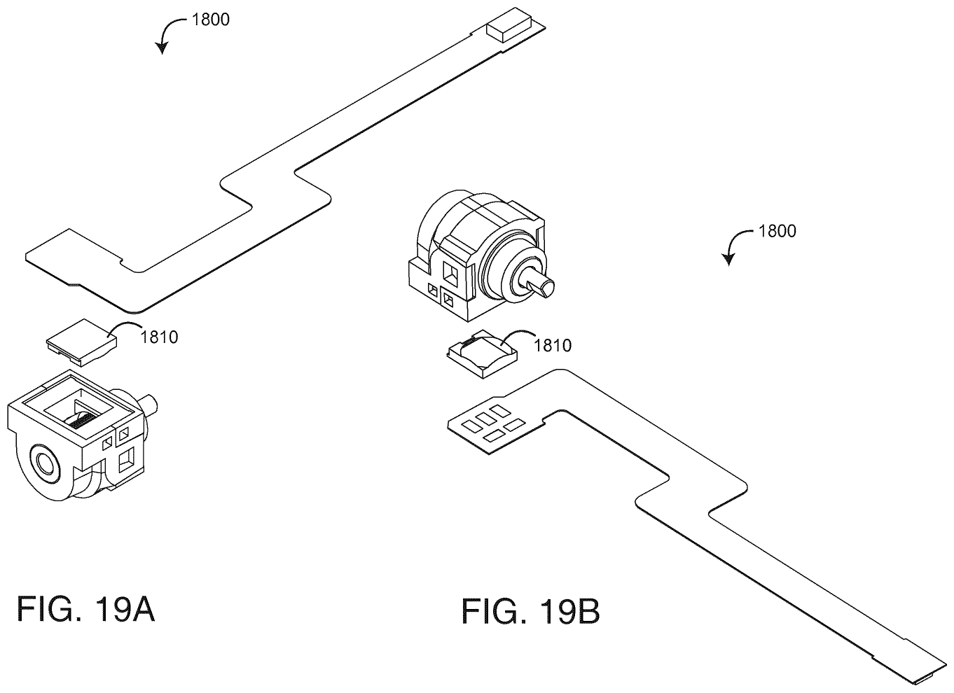

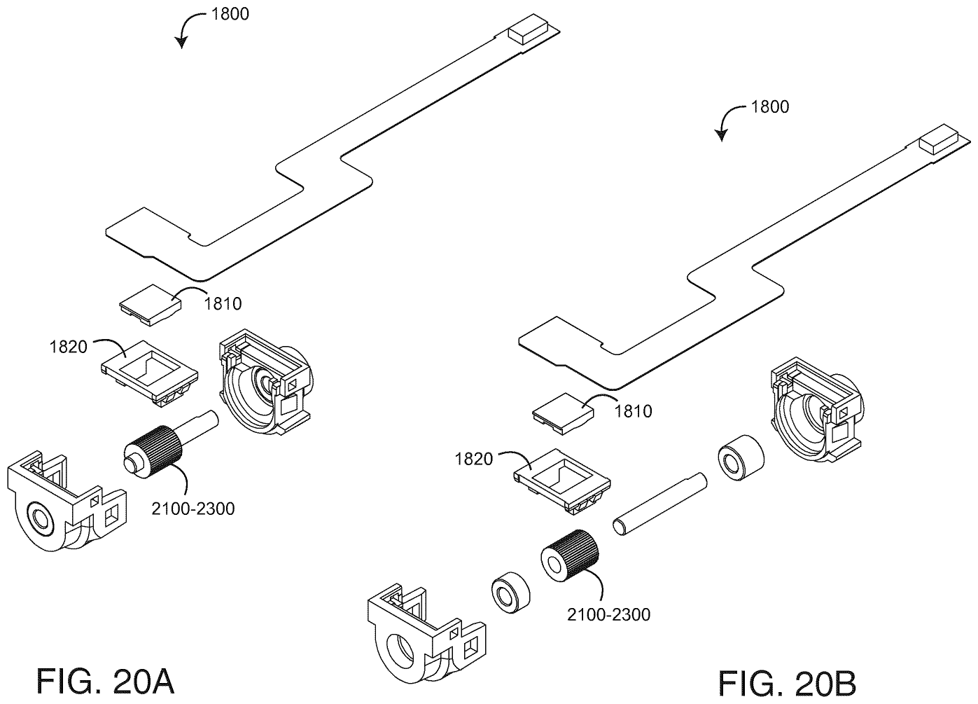

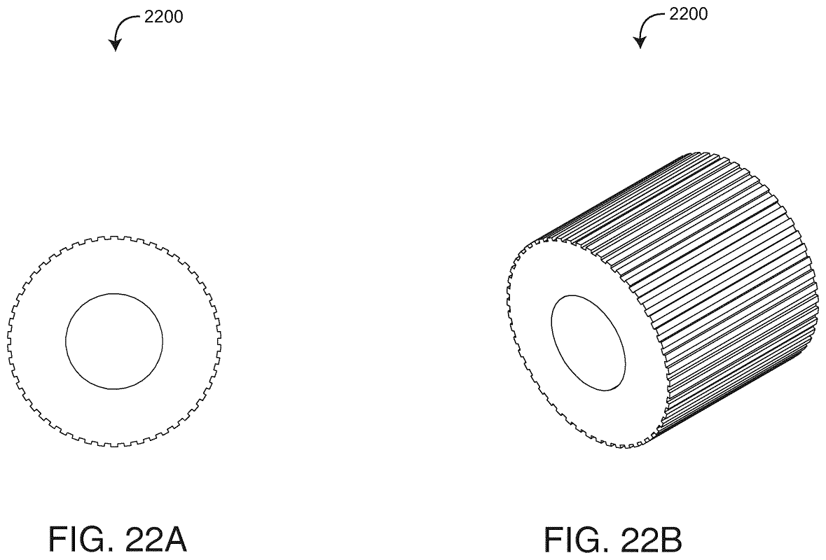

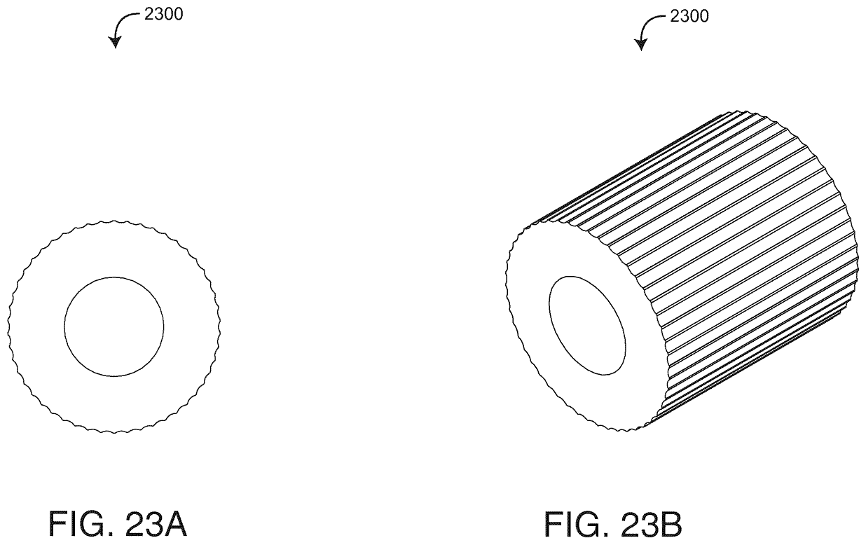

FIGS. 3-23 illustrate three position-encoder embodiments. Each of these embodiments advantageously utilize a double-bearing axle to stably mount an optical encoding device for the most precise optical measurements of the axle angular position and, hence, the linear position versus time of a pulser 140 (FIG. 1). In this manner, a precisely measured and controlled sensor active pulse can be generated.

FIGS. 3A-B generally illustrate slotted-wheel, position-encoder 301, 302 embodiments. The encoders 301, 302 each have an axle 310 with a double-bearing 320 mount to a housing 330. The slotted wheel 370 is mounted to the axle 310. LEDs 340 illuminate a wheel obverse side and detectors 350 sense the illumination through wheel slots on a wheel reverse side. A folded, slotted mask 361 is positioned on both sides of the slotted wheel 370 so that mask slots align with wheel slots at discrete axle positions. Accordingly, axle position pulses are generated as the axle 310 rotates the wheel 340 and the wheel slots alternately block and pass light, as generated and sensed with the LED/detector optics 340, 350.

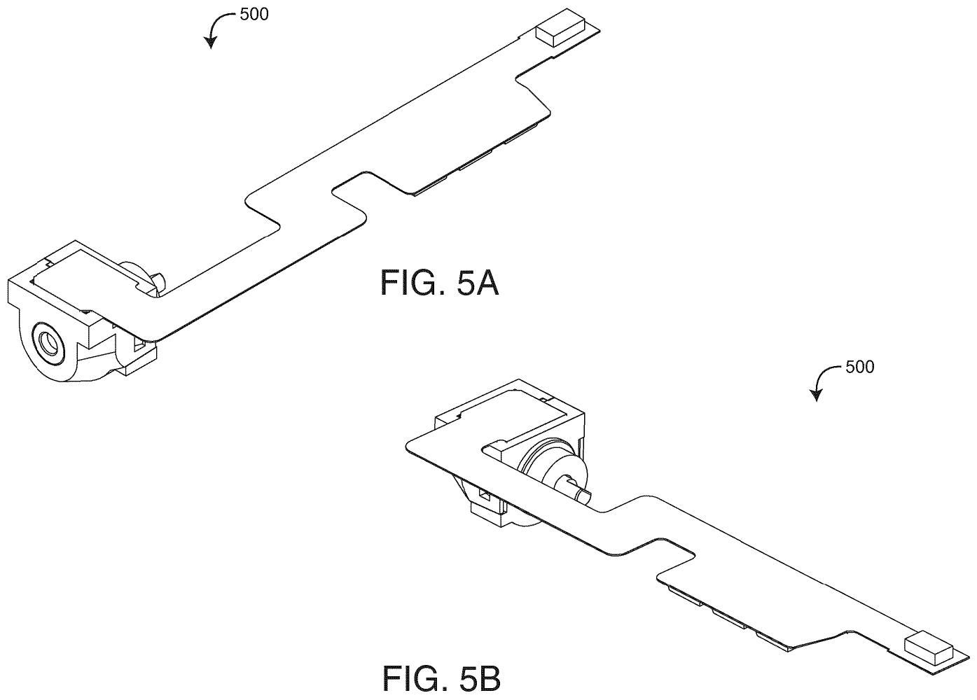

As shown in FIG. 3A, the LED/detector optics 340, 350 are located perpendicular to the slotted wheel, and the mask 361 is reflective. A slotted wheel position encoder embodiment according to FIG. 3A is described in detail with respect to FIGS. 5-13, below.

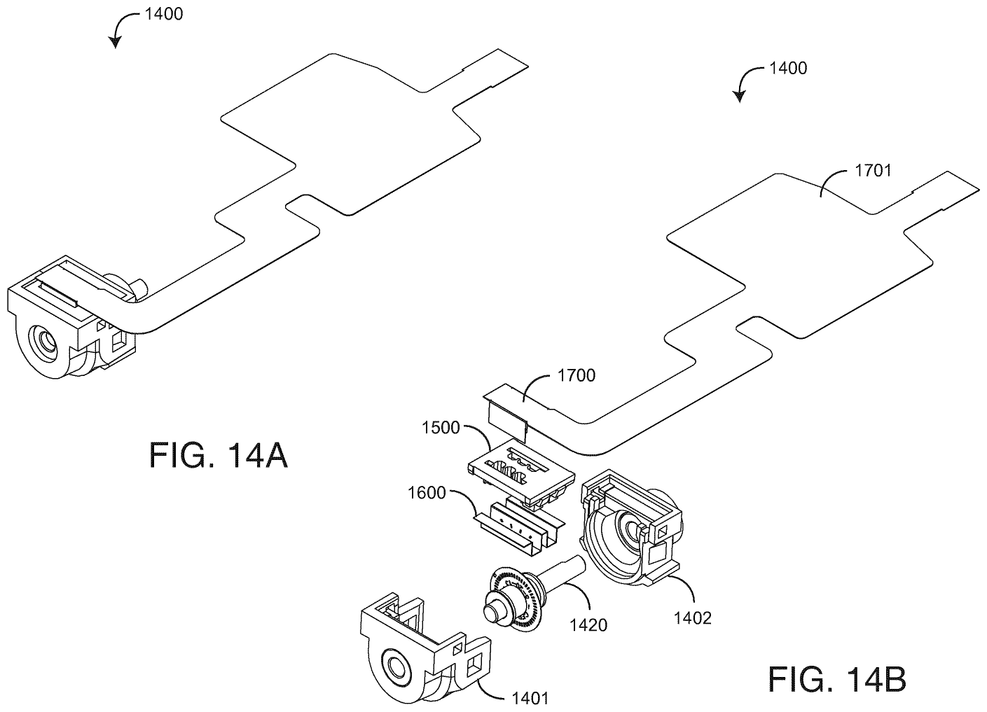

As shown in FIG. 3B, the LED/detector optics 340, 350 are located parallel to the slotted wheel so as to directly illuminate and sense via the mask 362. A slotted wheel position encoder embodiment according to FIG. 3B is described in detail with respect to FIGS. 14-17, below.

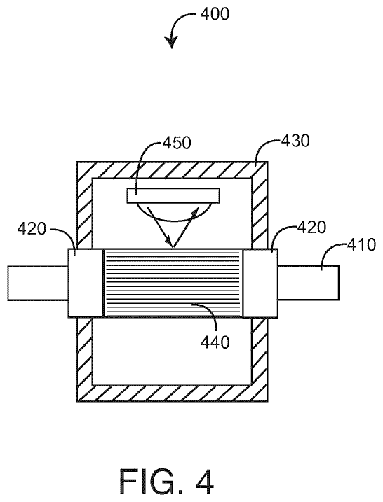

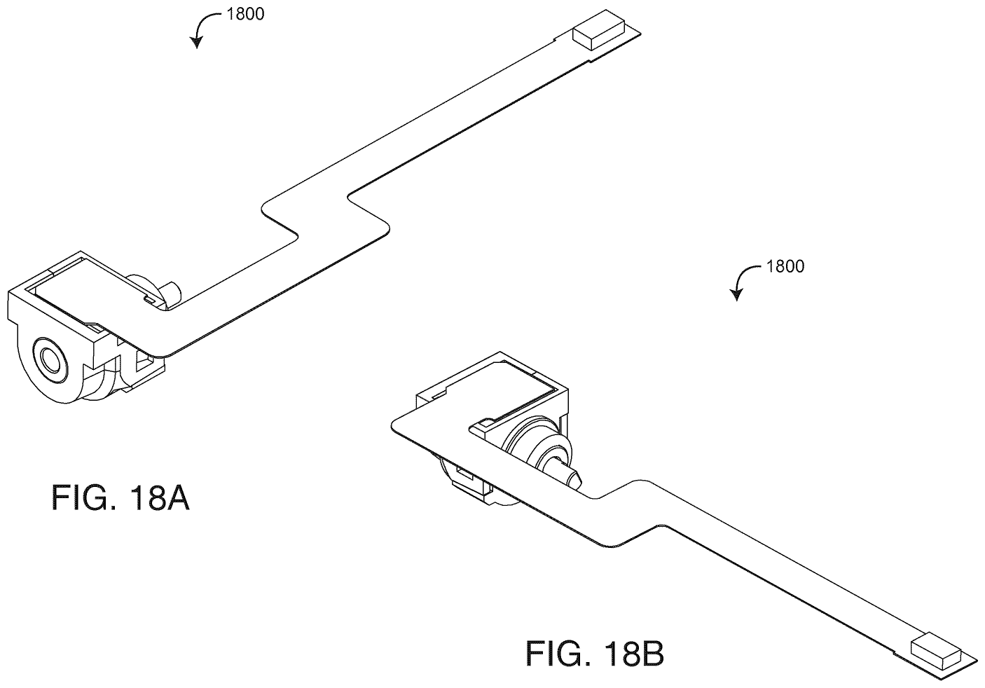

FIG. 4 generally illustrates a reflective-cylinder, position-encoder 400 embodiment. The encoder 400 has an axle 410 with a double-bearing 420 mount to a housing 430. A reflective cylinder 440 is mounted to the axle 410. The cylinder surface has a repetitive reflective structure disposed across the length of the cylinder. A commercial optical encoder 450 is located over the cylinder so as to sense the reflective structure 440 and determine axle position accordingly. In an embodiment, the optical encoder is a 3-channel reflective incremental encoder available from Avago Technologies, San Jose, Calif. A reflective cylinder position encoder embodiment according to FIG. 4 is described in detail with respect to FIGS. 18-23, below.

### Slotted Wheel Encoder—Indirect Illumination Encoder Mask

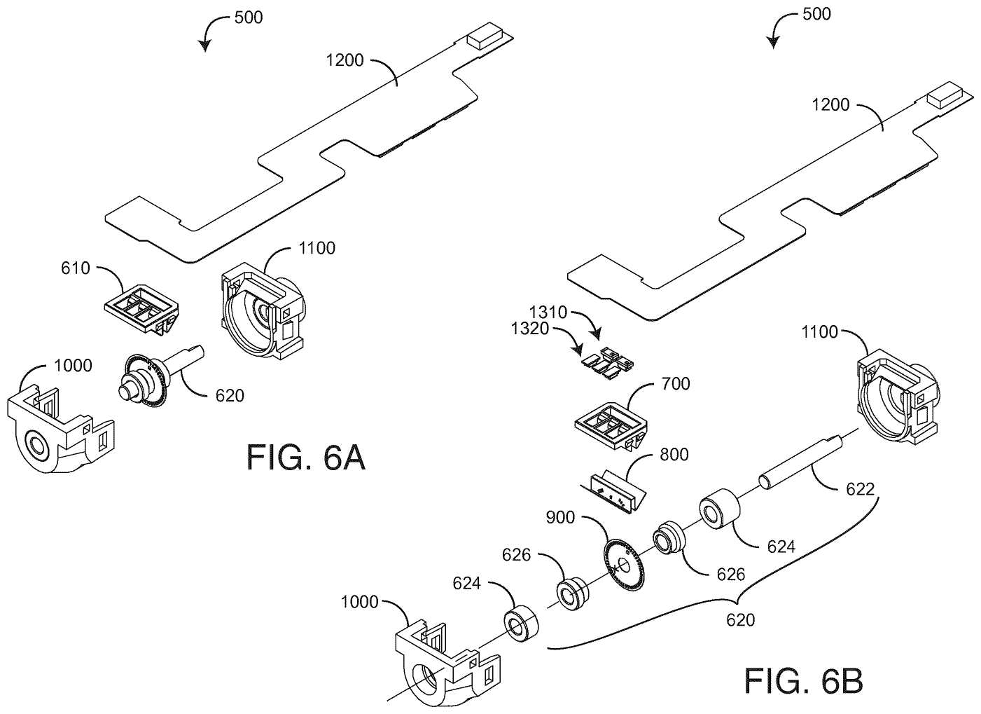

FIGS. 5-13 illustrate details of a double-bearing, slotted-wheel, position-encoder embodiment utilizing an indirectly-illuminated (indirect) encoder mask. FIGS. 5-6 illustrate the double-bearing position encoder 500 assembly which reads an encoder wheel 900 via a wheel-edge-mounted photo interrupter 610. The encoder wheel 900 is part of an encoder assembly 620. The encoder assembly 620 is advantageously mounted within a double-bearing encoder housing 1000, 1100. The photo interrupter 610 includes an encoder mask block 700 that houses a reflective encoder mask (origami) 800, LEDs 1310 and detectors 1320. The LEDs 1310 and detectors 1320 are mechanically mounted to, and in electrical communications with, a flex circuit 1200 that generates LED 1310 drive signals and receives and processes detector 1320 signals. The encoder assembly 620 has an encoder wheel 900 mounted between encoder wheel bushings 626 and shaft bushings 624. The photo interrupter 610 is mounted onto the encoder housing 1000, 1100 over an encoder wheel 900 edge.

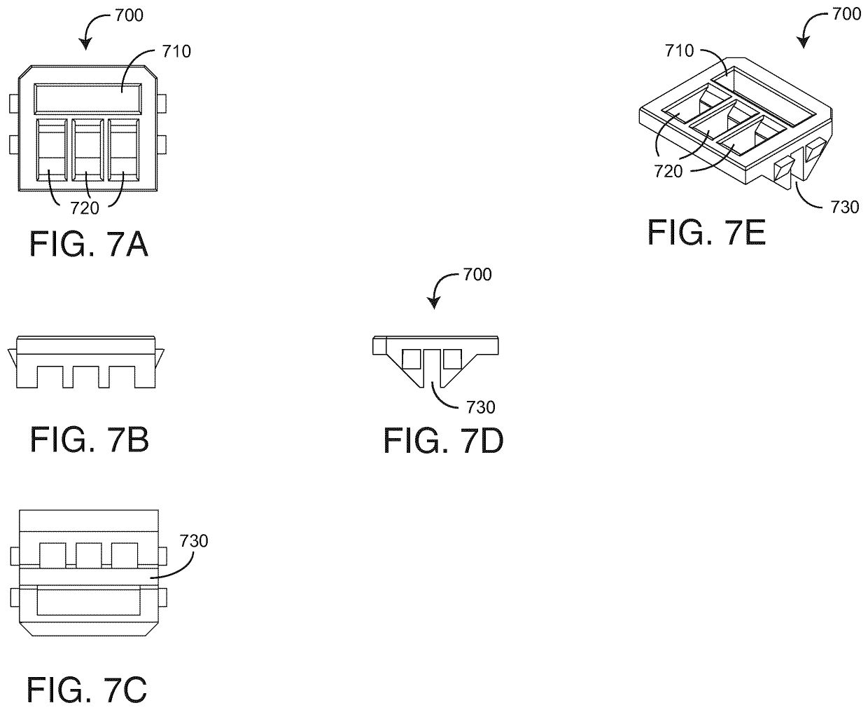

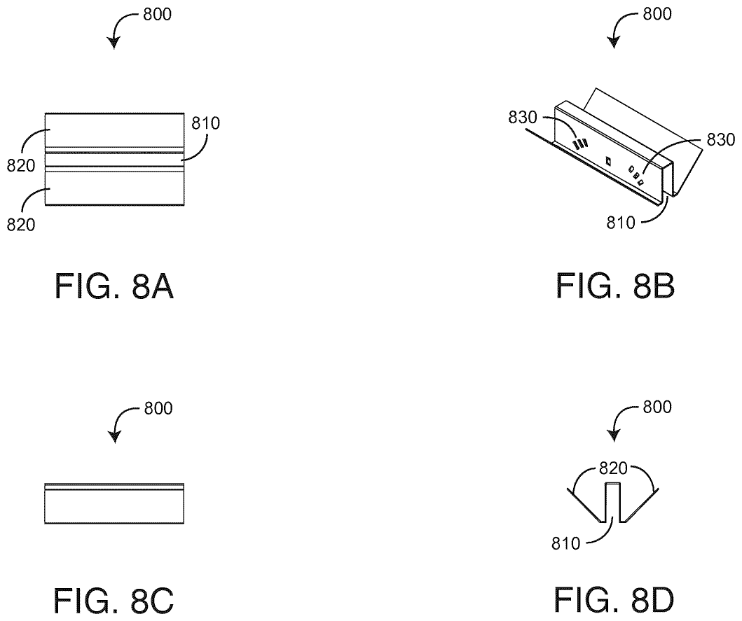

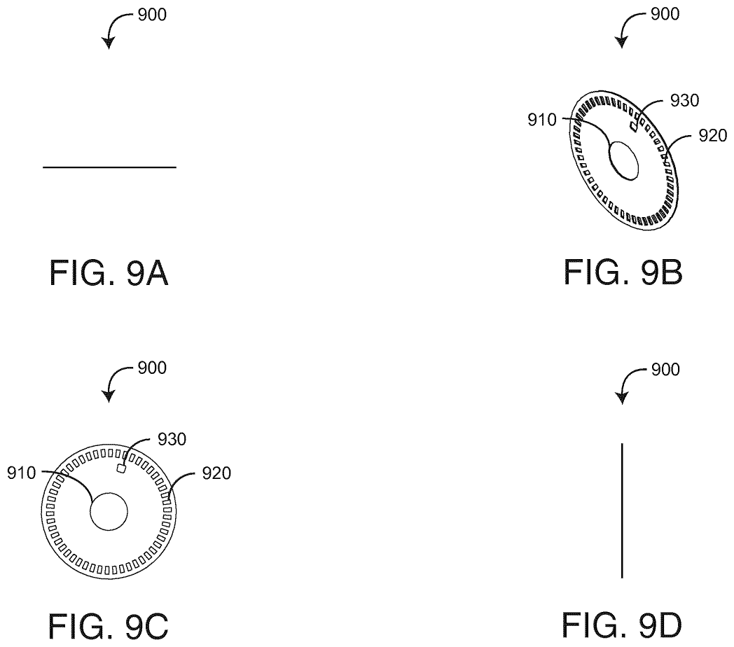

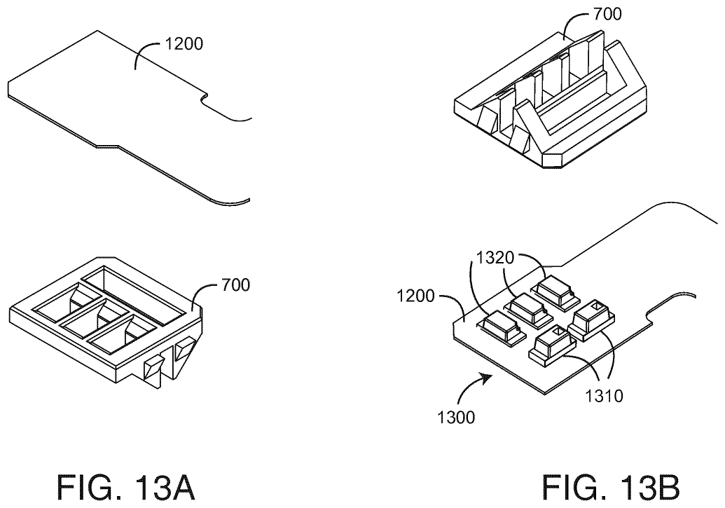

FIGS. 7A-E illustrate an encoder mask block 700 that houses the flex circuit-mounted optics 1310, 1320 (FIGS. 13A-B) proximate to the encoder mask 800 (FIGS. 8A-D). FIGS. 8A-D illustrate the encoder mask 800, which defines an encoder wheel path 810, reflective surfaces 820 and mask slots 830. The encoder mask allows the LEDs/detectors 1310, 1320 (FIG. 13B) to read the wheel slots at 0 and 90 electrical degrees. In particular, LED 1310 (FIG. 13B) light is reflected off one surface 820 through the slots 830 and intermittently through the encoder slots 920 as the encoder 900 spins within the wheel path 810. The intermittent light is reflected off another surface 820 to the detectors 1320 (FIG. 13B). FIGS. 9A-D illustrate a slotted encoder wheel 900 constructed as a thin, round disk defining a center-mount hole 910, encoder slots 920 and an index slot 930.

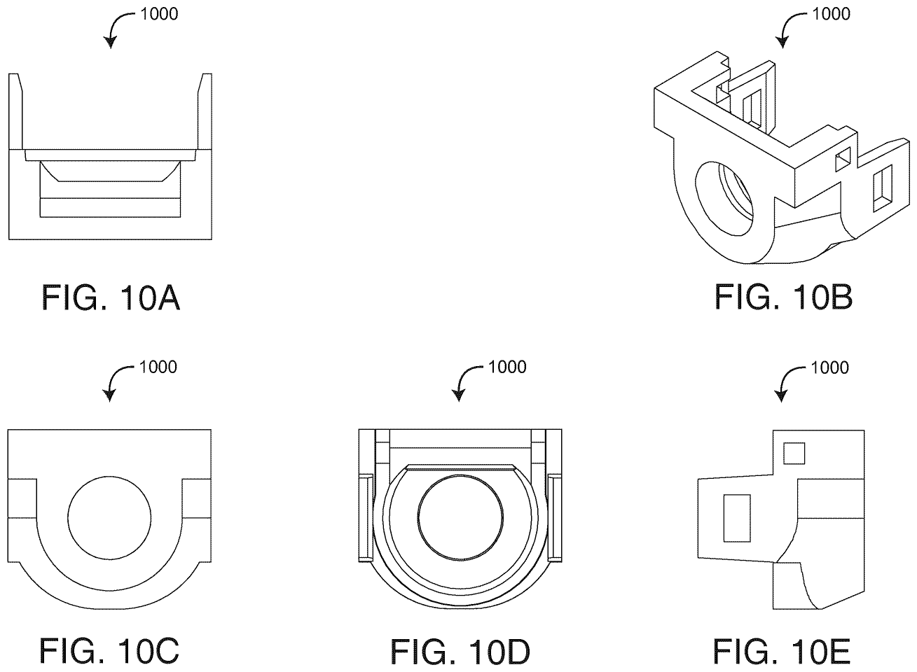

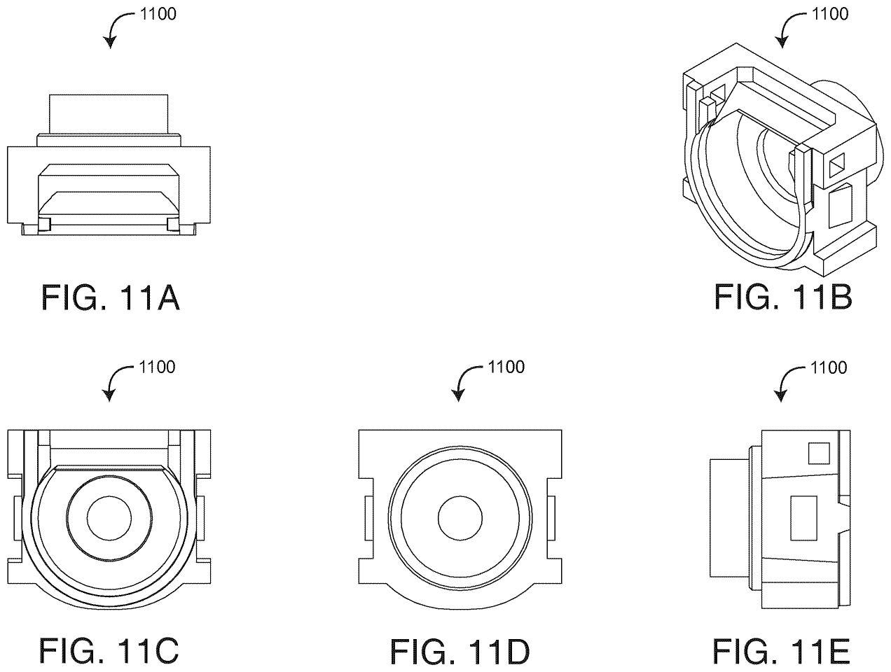

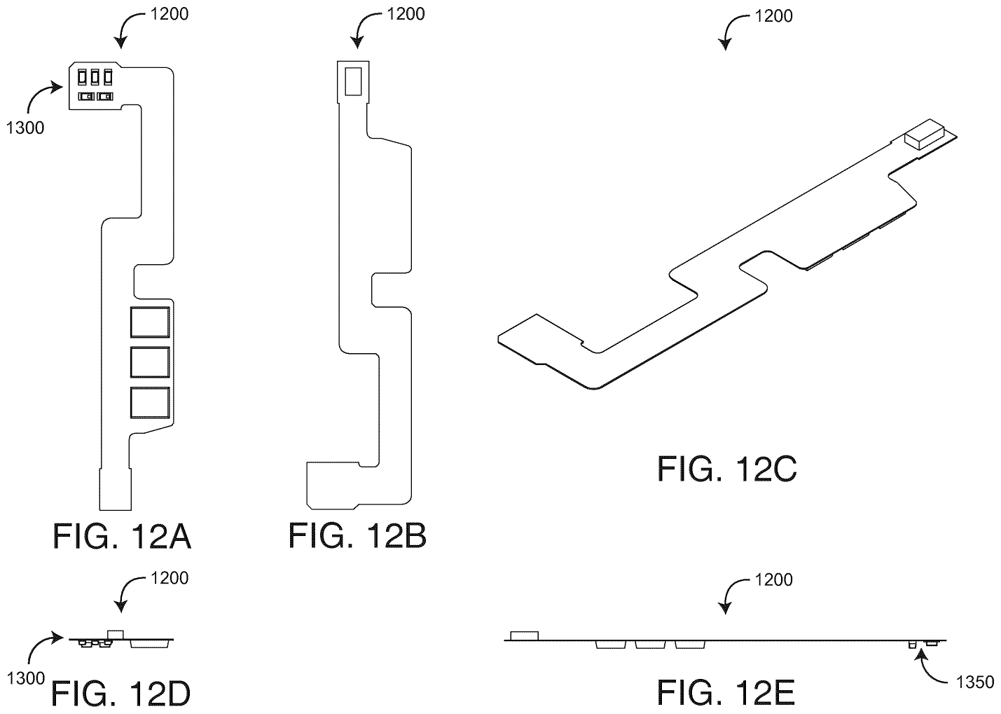

FIGS. 10-11 illustrate the encoder front housing 1000 and back housing 1100 that advantageously provides a double-bear mount for the encoder assembly 620 (FIGS. 6A-B). Further the housing 1000, 1100 positions the photo interrupter 610 (FIGS. 6A-B) over the encoder wheel 900 so as to detect the passing encoder slots 920 (FIGS. 9A-D). FIGS. 12-13 illustrate the encoder flex circuit assembly 1200 and corresponding optics 1300 and mask block 700, which generate signals responsive to the encoder 900 (FIGS. 9A-D) position as it rotates in response to a shaft-coupled, motor-driven active pulser 110, 120, 140 (FIG. 1).

### Slotted Wheel Encoder—Direct Illumination Mask

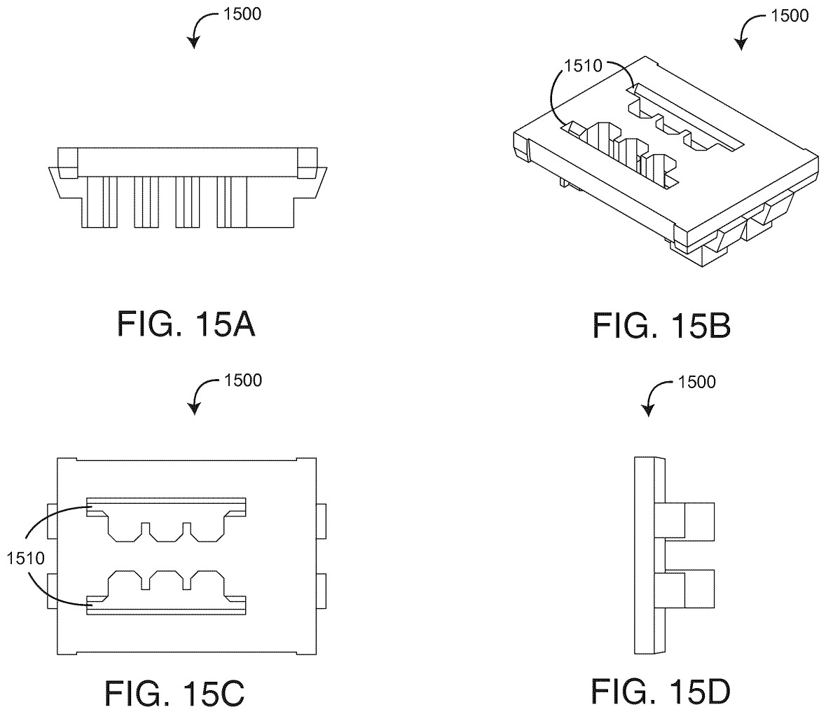

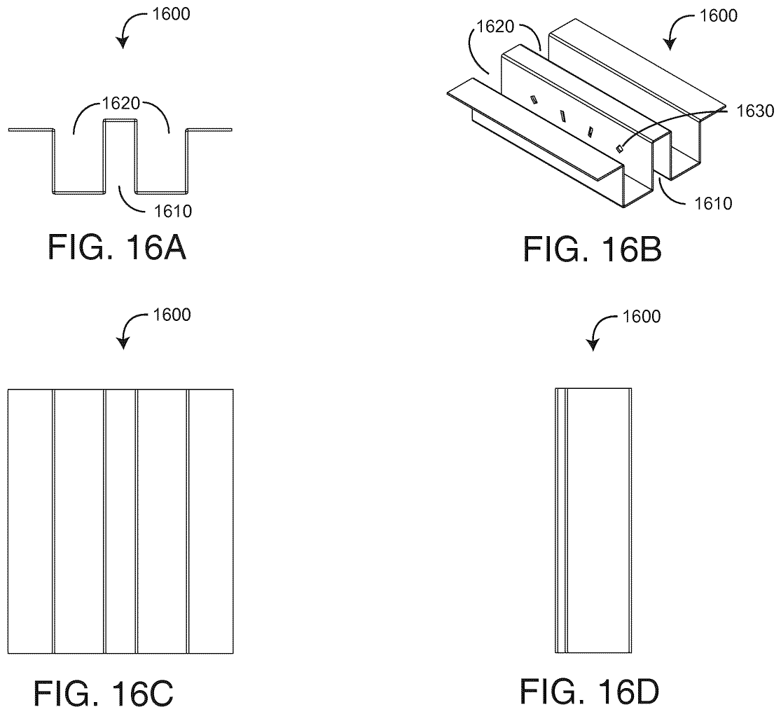

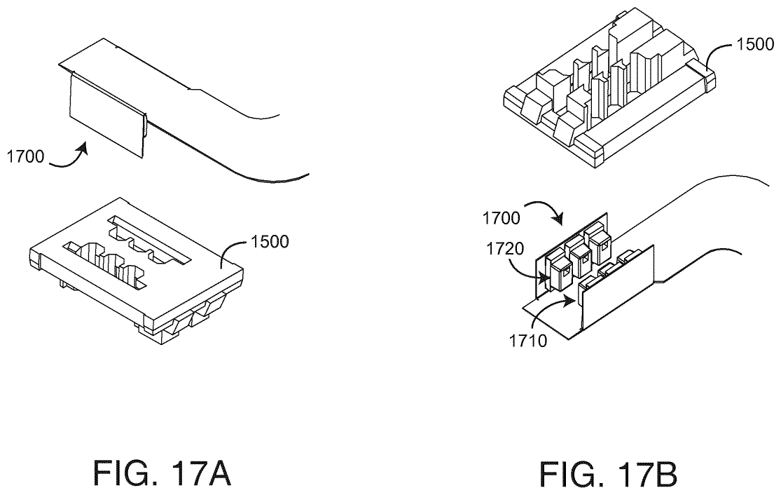

FIGS. 14-17 illustrate details of a double-bearing, slotted-wheel, position-encoder 1400 embodiment utilizing a direct illumination encoder mask. FIGS. 15A-D illustrate an encoder mask block 1500 that positions flex circuit-mounted optics to the mask 1600 (FIGS. 16A-D). FIGS. 16A-D illustrate the encoder mask origami 1600 having mask slots for reading the wheel slots at 0 and 90 electrical degrees. FIGS. 17A-B illustrate flex circuit optics 1700 and the corresponding encoder mask block 1500 (FIGS. 15A-D).

As shown in FIGS. 14A-B, a double-bearing position encoder 1400 assembly reads an encoder wheel portion of an encoder assembly 1420 via a wheel-edge-mounted direct illumination mask 1600 and proximate-mounted LED/detector optics 1700 (FIGS. 17A-B). The encoder assembly 1420 is advantageously mounted within a double-bearing encoder housing 1401, 1402. A photo interrupter includes an encoder mask block 1500 that houses a direct illumination encoder mask 1600, LEDs 1710 (FIG. 17B) and detectors 1720 (FIG. 17B). The LEDs and detectors are mechanically mounted to, and in electrical communications with, a flex circuit 1701 that generates LED drive signals and receives and processes detector signals. The encoder assembly 1420 has an encoder wheel mounted between encoder wheel bushings and shaft bushings as described above. The photo interrupter 1500, 1600 is mounted onto the encoder housing 1401, 1402 over an encoder wheel edge.

FIGS. 15A-D illustrate an encoder mask block 1500 that houses the flex circuit-mounted optics 1710, 1720 (FIG. 17B) proximate to the encoder mask 1600 (FIGS. 16A-D). FIGS. 16A-D illustrate the encoder mask 1600, which defines an encoder wheel path 1610, a direct optical path 1620 and mask slots 1630. The encoder mask allows the LEDs/detectors 1710, 1720 (FIG. 17B) to read the wheel slots at 0 and 90 electrical degrees. In particular, LED 1710 (FIG. 13B) light is directly transmitted 1620 through the slots 1630 and intermittently through the encoder slots 920 (FIG. 9B) as the encoder spins within the wheel path 1610. The intermittent light is directly transmitted 1620 to the detectors 1720 (FIG. 17B).

### Reflective Cylinder Encoder

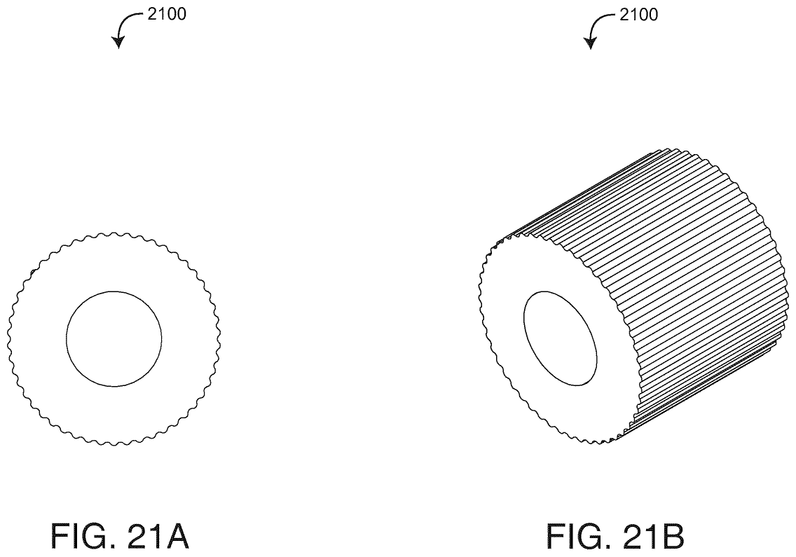

FIGS. 18-23 illustrate details of double-bearing, reflective cylinder, position-encoder 1800 embodiment utilizing an off-the-shelf reflective encoder 1810 mounted proximate a double-bearing reflective encoder cylinder 2100-2300 (FIGS. 21-23). FIGS. 18-20 illustrate the double-bearing position encoder 1800 embodiment having an off-the-shelf reflective encoder 1810, an encoder block 1820 and a reflective encoder cylinder 2100-2300. FIGS. 21-23 illustrate various encoder cylinder embodiments.

A double-bearing position encoder has been disclosed in detail in connection with various embodiments. These embodiments are disclosed by way of examples only and are not to limit the scope of the claims that follow. One of ordinary skill in the art will appreciate many variations and modifications.

1. (canceled)

2. An apparatus for non-invasive physiological monitoring comprising:
an axle disposed within a housing and supported by a plurality of bearings; and
wherein rotation of the axle causes generation of an active pulse at a tissue site for analysis by an optical sensor;
an encoder coupled to the axle and comprising a first opening, wherein the rotation of the axle further causes rotation of the encoder;
a light source configured to emit light;
an encoder mask comprising a plurality of openings and extending over an edge of the encoder and at least partially along a first side, and a second side of the encoder, wherein the plurality of openings comprises at least a second opening, and wherein the second opening is configured to allow detection of the emitted light during rotation of the encoder after the emitted light passes through the opening of the encoder; and
a detector configured to detect light emitted by the light source after the light passes through the first opening and the second opening and reflects off a portion of the encoder mask at least one time, wherein the detector is further configured to generate a signal responsive to the detected light, wherein the signal is indicative of at least one of a parameter associated with the active pulse.

3. The apparatus of claim 2, wherein the parameter associated with the active pulse is a frequency of the active pulse.

4. The apparatus of claim 2, wherein the parameter associated with the active pulse is a phase of the active pulse.

5. The apparatus of claim 2, wherein the light reflects off of the portion of the encoder mask prior to passing through at least one of the first opening or the second opening.

6. The apparatus of claim 2, wherein the light reflects off of the portion of the encoder mask after passing through at least one of the first opening or the second opening.

7. The apparatus of claim 2, wherein the light reflects off of a first portion of the encoder mask prior to passing through the first opening, and wherein the light reflects off of a second portion of the encoder mask after passing through the first opening and prior to reaching the detector.

8. The apparatus of claim 2, wherein the first opening comprises any one of a plurality of openings of the encoder.

9. The apparatus of claim 2, wherein light emitted from the light source reflects off the encoder mask prior to passing through the first opening during rotation of the encoder, and wherein the light reflects off the encoder mask prior to passing through the second opening to allow detection of light at a first phase respective to the emitted light.

10. The apparatus of claim 2, wherein the encoder mask comprises a third opening, and wherein the light reflects off the encoder mask prior to passing through the third opening to allow detection of light at a second phase respective to the emitted light.

11. A method for non-invasive physiological monitoring comprising:
receiving a signal that is responsive to light detected by a detector after the light passes through a first opening of an encoder, passes through a second opening of an encoder mask, wherein the second opening comprises any one of a plurality of openings of the encoder mask and wherein the second opening is configured to allow detection of light passing through the first opening during rotation of the encoder, and reflects off a portion of the encoder mask at least one time, wherein the encoder is coupled to an axle disposed within a housing and supported by a plurality of bearings, wherein rotation of the axle causes generation of an active pulse at a tissue site for analysis by an optical sensor; wherein the rotation of the axle further causes rotation of the encoder; and
determining a parameter associated with the active pulse based at least in part on the received signal.

12. The method of claim 11, further comprising determining a position of the encoder based at least in part on the signal, wherein said determining the parameter is based at least in part on the position of the encoder.

13. The method of claim 11, wherein the parameter associated with the active pulse is a frequency of the active pulse.

14. The method of claim 11, wherein the parameter associated with the active pulse is a phase of the active pulse.

15. The method of claim 11, wherein the light reflects off of the portion of the encoder mask prior to or after passing through at least one of the first opening or the second opening and prior to reaching the detector.

16. The method of claim 11, wherein the light reflects off of a first portion of the encoder mask prior to passing through the opening of the encoder, and wherein the light reflects off of a second portion of the encoder mask after passing through the opening of the encoder and prior to reaching the detector.

17. A noninvasive optical sensor comprising:
an axle disposed within a housing and supported by a plurality of bearings, wherein rotation of the axle causes generation of an active pulse at a tissue site for analysis by the optical sensor;
an encoder coupled to the axle and comprising a first opening, wherein the rotation of the axle further causes rotation of the encoder;
a light source configured to emit a first light through the first opening;
an encoder mask comprising a plurality of openings and extending at least partially along a first side, a second side, and an edge of the encoder, wherein a second opening of the plurality of openings on the encoder mask is configured to allow detection of the first light after the first light passes through the first opening during rotation of the encoder;
a first detector configured to detect the first light emitted by the first light source after the first light passes through the first opening and the second opening and reflects off a portion of the encoder mask at least one time, wherein the first detector is further configured to generate a first signal responsive to the detected first light, wherein the first signal is indicative of a parameter associated with the active pulse; and
a second detector configured to detect second light after the second light is attenuated by tissue of an individual, wherein the second detector is configured to generate a second signal that is responsive to the detected second light, wherein the second signal is indicative of a physiological parameter associated with the individual.

18. The noninvasive optical sensor of claim 17, wherein the parameter associated with the active pulse is a frequency of the active pulse.

19. The noninvasive optical sensor of claim 17, wherein the parameter associated with the active pulse is a phase of the active pulse.

20. The noninvasive optical sensor of claim 17, wherein the first light reflects off of the portion of the encoder mask prior to or after passing through at least one of the first opening or the second opening

21. The noninvasive optical sensor of claim 17, wherein the first light reflects off of a first portion of the encoder mask prior to passing through the first opening, and wherein the first light reflects off of a second portion of the encoder mask after passing through the first opening and prior to reaching the first detector.
